(19) United States
(12) Patent Application Publication
(10) Pub. No.: US 2019/0226005 A1
(43) Pub. Date: Jul. 25, 2019

(54) Multidifferential Agar with Chromogenic Substrates

(71) Applicant: S2M Enterprises, LLC, Spokane Valley, WA (US)

(72) Inventors: Molly Paridon, Spokane, WA (US); Sommer Teague, Spokane Valley, WA (US); Stephanie Bernards, Spokane, WA (US)

(21) Appl. No.: 15/875,580

(22) Filed: Jan. 19, 2018

(51) Int. Cl. C12Q 1/04

(52) U.S. Cl. CPC C12Q 1/045; G01N 2333/255; G01N 2333/245

(57) ABSTRACT

Multidifferential agar for differentially detecting Bile-Tolerant Gram-Negative organisms, E. coli, Salmonella, and non-E. coli coliforms and methods for using the same are disclosed. The multidifferential agar may include chromogenic substrate(s), agar, gelatin, an inhibitor of gram-positive bacteria, bacterial nutrients, propylene glycol, surfactants, and/or bile salts.

### BACKGROUND

Microbial contamination of products, such as nonsterile drugs or cosmetics, can be a major threat to public health. Products that are not sterilized may contain certain microorganisms such as Escherichia coli or Salmonella, which can be hazardous even when present in low amounts. To protect consumers, government agencies may require manufacturers of certain products to evaluate the presence of microorganisms in their products. Typically, microbial examination of a product is performed by applying a sample of the product to multiple agar plates, which are each used to detect organisms, such as Escherichia coli, Salmonella, or coliforms. These methods typically include culturing the sample in a liquid nutrient broth for several hours to several days and then incubating the resulting culture on agar plates for an additional period of time. Multiple culturing steps may be required, and the process may take several days.

### BRIEF DESCRIPTION OF THE DRAWINGS

The detailed description is set forth below with reference to the accompanying figures. In the figures, the left-most digit(s) of a reference number identifies the figure in which the reference number first appears. The same reference numbers in different figures indicate similar or identical items. The components depicted in the accompanying figures are not to scale and components within the figures may be depicted not to scale with each other.

FIG. 1 illustrates an example process for detecting Bile-Tolerant Gram-Negative (BTGN) organisms, Escherichia coli, Salmonella species, and non-E. coli coliforms (including Klebsiella and Citrobacter) on a multidifferential agar plate.

FIG. 2 illustrates another example process for detecting Salmonella on a multidifferential agar plate.

FIG. 3 illustrates a flow diagram of an example process for the of use of a multidifferential agar to detect BTGN organisms, E. coli, and Salmonella ssp., and/or non-E. coli coliforms.

FIG. 4 illustrates a flow diagram of another example process for the of use of a multidifferential agar with propylene glycol to differentially detect Salmonella.

FIG. 5 illustrates an example process for producing multidifferential agar plates.

FIG. 6 illustrates a workflow to test for the presence of BTGN, as outlined in United States Pharmacopeia (USP) General Chapters <61> and <62>.

FIG. 7 illustrates a workflow to test for the presence of E. coli, as outlined in United States Pharmacopeia (USP) General Chapters <61> and <62>.

FIG. 8 illustrates a workflow to test for the presence of Salmonella, as outlined in United States Pharmacopeia (USP) General Chapters <61> and <62>.

FIGS. 9A-9C illustrate results from a comparison study using a multidifferential agar versus the traditional medium outlined in United States Pharmacopeia (USP) General Chapters <61> and <62>, which specifically include the tests for E. coli, Salmonella, and BTGN organisms.

FIGS. 10A-C illustrate results for detecting and enumerating E. coli utilizing the multidifferential agars and methods described herein.

FIGS. 11A-C illustrate additional results for detecting and enumerating E. coli utilizing the multidifferential agars and methods described herein.

FIGS. 12A-C illustrate results for detecting and enumerating Pseudomonas aeruginosa utilizing the multidifferential agars and methods described herein.

FIGS. 13A-C illustrate results for detecting and enumerating Salmonella typhimurium utilizing the multidifferential agars and methods described herein.

FIG. 14 illustrates inhibition of growth of Staphylococcus aureus on multidifferential agar and on the compendial agars Violet Red Bile Glucose and MacConkey.

### DETAILED DESCRIPTION

Multidifferential agar for the detection and enumeration of Escherichia coli, Salmonella, non-E. coli coliforms and/or BTGN organisms, and methods for using the multidifferential agar are described herein. Microbial contamination can be a problem, for example, non-sterile medications. Therefore, testing ingredients for microbial contamination is performed. The United States Pharmacopeia (USP) has set recommended microbial limits based upon the article and route of administration. Many raw materials and finished products require testing for E. coli, Salmonella spp., and BTGN organisms. Current methods testing for microbial contamination can include incubating cultures taken from a product sample, utilizing enrichment broths, and plating the cultures onto multiple selective, differential agar plates or agar thin films, which may be used to detect a bacterial species/population of interest. Protocols for testing raw materials or products for E. coli, non-E. coli coliforms, Salmonella, and BTGN organisms may include homogenizing a sample, rinsing the sample in a diluent, and inoculating multiple nutrient broths with a subset of the diluted sample. Each broth may be intended to propagate a subset of organisms prior to plating the brothed samples onto multiple agars to detect, and optionally enumerate, specific organisms. The agar may be incubated for up to a week in order to detect whether the specific organisms of interest are present. The current methods for differentially detecting non-E. coli coliforms, BTGN organisms, Salmonella, and E. coli typically take at least three days to complete and require multiple enrichment broths, incubation temperatures, sample manipulations, and agar plates for each sample.

The innovation disclosed herein provides a rapid, one-plate solution for more accurately detecting multiple microbial contaminants, saving time and supplies. The plates may include a base agar that inhibits growth of unwanted microorganisms, and at least three chromogenic substrates, allowing for differential detection of more than one type of bacteria on the same plate. In examples, the multidifferential agar described herein may be used to differentially detect E. coli, Salmonella, non-E. coli coliforms, and/or Bile-Tolerant Gram-Negative organisms using a single plate, which may include at least three of the chromogenic substrates X-Caprylate, Magenta-Caprylate, Magenta-Gal, Salmon-Gal/Rose-Gal, Green-ß-D-Gal, Red-Gal, Purple-ß-D-Gal, BCIG, Indoxyl-ß-glucoside, or X-Glucoside.

It should be understood that while examples used herein describe multidifferential agars utilized for the detection of specific microbial contaminants present in a botanical sample, additional uses of the multidifferential agars other than in the use of botanical microbial testing are included in this disclosure. For example, the multidifferential agars described herein may be used to test for bacterial contaminants (e.g., Salmonella, and/or Pseudomonas) present in nonsterile drugs (e.g., a topical ointment) and/or cosmetics. Additionally, or alternatively, it should be understood that the multidifferential agars described herein may be utilized to test for microbial contaminants other than the specific example microbial contaminants listed and/or discussed in this disclosure.

The present disclosure provides an overall understanding of the principles of the composition, function, and use of the multidifferential agars and methods disclosed herein. One or more examples of the present disclosure are illustrated in the accompanying drawings. Those of ordinary skill in the art will understand that the multidifferential agars and methods specifically described herein and illustrated in the accompanying drawings are non-limiting embodiments. The features illustrated or described in connection with one embodiment may be combined with the features of other embodiments, including as between multidifferential agars and methods of using the agars. Such modifications and variations are intended to be included within the scope of the appended claims.

Additional details are described below with reference to examples and embodiments.

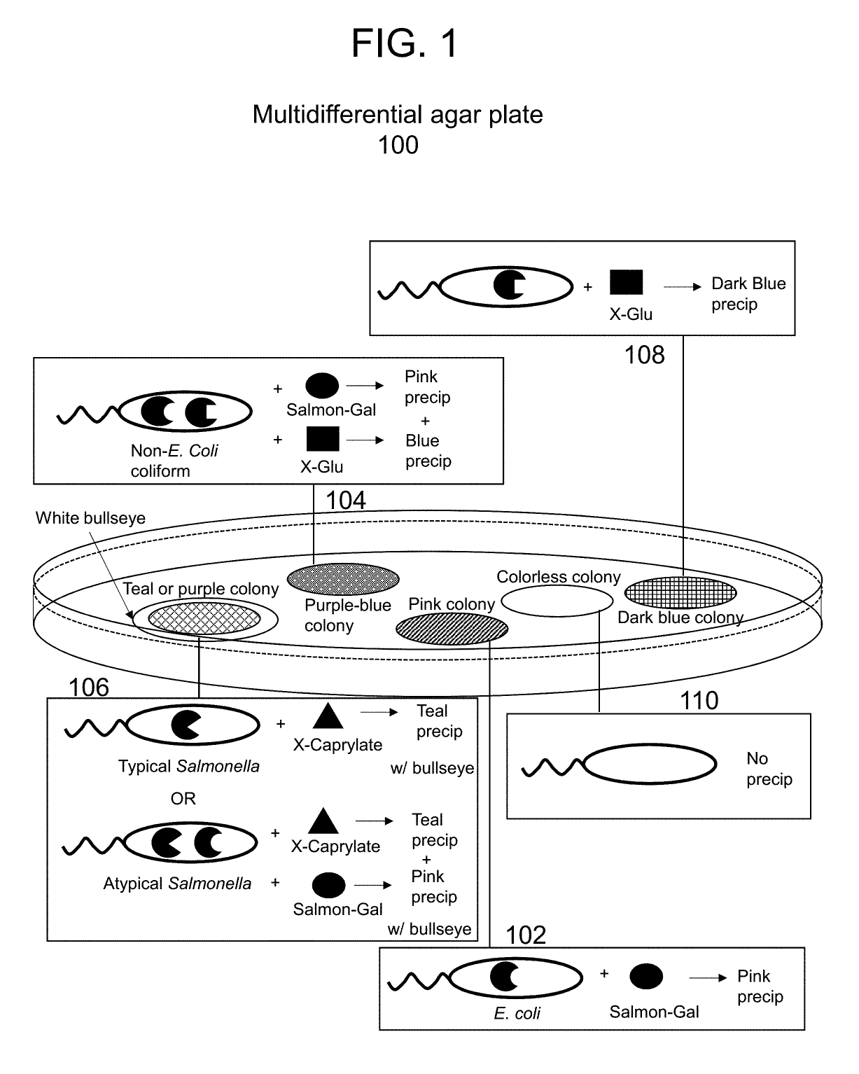

FIG. 1 illustrates an example of using a multidifferential agar 100 for differentially detecting E. coli, Salmonella, non-E. coli coliforms, and Bile-Tolerant Gram-Negative (BTGN) organisms.

The multidifferential agar 100 may include one or more chromogenic substrates. Chromogenic substrates are peptides that react with proteolytic enzymes and are converted to insoluble, colored precipitant. They are synthetic and designed to mimic selectivity of the natural substrate for the enzyme of interest. A chemical group is attached to the peptide portion of the substrate, which is released after enzyme cleavage, resulting in color formation. The resulting color can be seen with the naked eye without the aid of specialized equipment. Chromogenic substrates may be selected based, at least in part on the reaction of specific microorganisms of interest and the resulting color formation. The chromogenic substrate included in the multidifferential agar may include a β-galactosidase chromogenic substrate, a ß-D-Glucosidase chromogenic substrate, and a C-8 esterase chromogenic substrate.

The multidifferential agar may be used to detect one or more colonies of E. coli 102. E. coli, a gram-negative rod, is a lactose-fermenting member of the family Enterobacteriaceae (i.e., a coliform). Like other coliforms, most E. coli express β-galactosidase. However, unlike other coliforms, most E. coli do not express ß-D-Glucosidase.

The precipitate that may be present in a colony of E. coli 102 on the multidifferential agar 100 may be due to hydrolysis of β-galactosidase chromogenic substrate (also referred to herein as Substrate A). Examples of chromogenic substrates that may be used as Substrate A include Green-ß-D-Gal, Salmon-Gal/Rose-Gal, Magenta-Gal, Red-Gal, BCIG/X-Gal, and Purple-ß-D-gal. Salmon-Gal may refer to 6-Chloro-3-indolyl-β-D-galactopyranoside, which results in pink-magenta pigment (Aₘₐₓ=540 nm). Magenta-Gal may refer to 5-Bromo-6-chloro-3-indoxyl β-D-galactopyranoside, which results in magenta-red pigment (Aₘₐₓ=565 nm) BCIG may refer to the molecule 5-bromo-4-chloro-3-indoxyl-ß-D-galatopyranoside, which results in indigo pigment (Aₘₐₓ=615 nm). Purple-ß-D-Gal may refer to 5-Iodo-3-indolyl-ß-D-galactopyranoside, which results in pink-purple pigment (Aₘₐₓ=575 nm). Red-ß-D-Gal may refer to 5-Bromo-6-chloro-3-indolyl-ß-D-galactopyranoside, which results in pink-red pigment (Aₘₐₓ=565 nm). Green-ß-D-Gal may refer to N-methylindolyl-ß-D-galactopyranoside, which results in green pigment (Aₘₐₓ=665 nm). Substrate A may refer to any of the molecules listed above, which may be hydrolyzed by β-galactosidase, through cleavage of the ß-glycosidic bond in D-lactose. The resulting galactose and 5-bromo-4-chloro-3-hydroxyindole spontaneously dimerize and become oxidized, resulting in a water-insoluble pigment. Substrate A may be present in the multidifferential agar 100 at a concentration of between about 0.0015% (w/v) and about 1.0% (w/v), between about 0.003% (w/v) and about 1.0% (w/v), between about 0.003% (w/v) and about 0.01% (w/v), or between about 0.013% (w/v) and about 0.028% (w/v), between about 0.015% (w/v) and about 0.030% (w/v), about 0.01% (w/v), about 0.015% (w/v), about 0.02% (w/v), or about 0.025% (w/v). The example shown in FIG. 1 includes Salmon-Gal as Substrate A.

The multidifferential agar may be used to detect one or more colonies of non-E. coli coliforms 104. Coliform refers to lactose-fermenting bacteria that are part of the family Enterobacteriaceae, which is a broad group within BTGN bacteria. Coliforms are not a taxonomic group of bacteria, but are a functionally-defined bacterial group, and are used in microbial testing as an indicator of sanitation. Coliforms possess β-galactosidase activity, which cleaves Substrate A. Additionally, coliforms other than E. coli typically express ß-D-Glucosidase, which causes precipitate to form in the presence of a ß-D-Glucosidase chromogenic substrate (also referred to herein as Substrate B).

The precipitate that may be present in a colony of non-E. coli coliforms 104 on the multidifferential agar 100 may be due to hydrolysis of Substrate A and Substrate B. Substrate B may refer to a ß-D-Glucosidase chromogenic substrate and may include X-Glucoside/X-Glu. X-Glucoside may refer to 5-Bromo-4-chloro-3-indolyl β-D-glucopyranoside, which is a histochemical substrate for ß-D-Glucosidase that upon cleavage, may result in the production an insoluble, indigo-blue precipitate (Aₘₐₓ=615 nm). The X-Glucoside may be present in the multidifferential agar 100 at a concentration of between about 0.0001% (w/v) and about 1.0% (w/v), or between about 0.0005% (w/v) and about 0.01% (w/v), or between about 0.001% (w/v) and about 0.035% (w/v), or between about 0.003% (w/v) and about 0.01% (w/v), or between about 0.003% (w/v) and about 0.01% (w/v), or between about 0.005% (w/v) and about 0.1% (w/v), about 0.001% (w/v), about 0.0015% (w/v), about 0.002% (w/v), or about 0.008% (w/v). The example shown in FIG. 1 includes Salmon-Gal as Substrate A and X-Glucoside as Substrate B. Together, hydrolysis of these chromogens results in light to dark purple-blue colonies for non-E. coli coliforms.

The multidifferential agar may be used to detect one or more colonies of Salmonella 106. There are over 2,000 known strains of Salmonella, which are typically rod-shaped, bile-resistant, gram-negative motile bacterium. Salmonella is estimated to be responsible for over 1 million foodborne illnesses in the US per year. Unlike E. coli and non-E. coli coliforms, Salmonella express the enzyme C8-esterase.

The precipitate within a colony of Salmonella 106 may be due to cleavage of a C-8 esterase chromogenic substrate (also referred to herein as Substrate C). Examples of substrates that can be used as Substrate C include X-Caprylate and Magenta Caprylate. X-Caprylate may refer to the molecule 5-Bromo-4-chloro-3-indoxyl caprylate, which yields a teal precipitate (Aₘₐₓ=615 nm). Magenta-Caprylate may refer to 5-bromo-6-chloro-3-indolyl caprylate, which yields a pink-magenta precipitate (Aₘₐₓ=565 nm.) In examples, Substrate C may be present in the multidifferential agar 100 at a concentration of between about 0.001% (w/v) and about 1.0% (w/v), between about 0.15 g/L and about 0.40 g/L, between about 0.017% (w/v) and about 0.032% (w/v), between about 0.01% (w/v) and about 4.0% (w/v), about 0.015% (w/v), about 0.02% (w/v), about 0.025% (w/v), about 0.016% (w/v), or about 0.032% (w/v). Lactose negative Salmonella do not express β-galactosidase, and therefore do not react with Substrate A. The example shown in FIG. 1 includes X-Caprylate as Substrate C. Salmonella colonies on a multidifferential agar 100 plate with X-Caprylate will typically be bright blue-green (aqua blue) with a unique bullseye/ring appearance. However, atypical species of Salmonella may express β-galactosidase as well as C8-esterase. Therefore, colonies of atypical Salmonella in the example shown in FIG. 1 may include a pink precipitate and a teal precipitate, which may result in purple-blue colonies with the unique bullseye ring appearance. The bullseye is only demonstrated on multidifferential agar 100 by Salmonella species. The white ring/bullseye surrounding a colony of Salmonella 106 on a multidifferential agar plate 100 may be due to the Salmonella metabolizing propylene glycol, leading to acidification of the environment immediately surrounding the colony and the appearance of a white ring or bullseye around the colony. The white ring may appear white, white-ish blue, or a lighter shade than the colony. The propylene glycol may be present in the multidifferential agar 100 at a concentration of between about 0.0001 and about 1%, or between about 0.001% and about 0.1%

Colonies of bile-tolerant gram-negative (BTGN) bacteria that express ß-D-Glucosidase, but not β-galactosidase nor C8-esterase 108 may include a precipitate due to the hydrolysis of Substrate B. In the example shown in FIG. 1, X-Glucoside is used as Substrate B, and therefore these colonies appear dark blue on the multidifferential agar.

Colonies of other bile-tolerant gram-negative (BTGN) 110 that do not express ß-D-Glucosidase, β-galactosidase, nor C8-esterase may not include a colored precipitate. These colonies may appear tan, beige, or colorless on the multidifferential agar 100.

In the multidifferential agar 100, the ratio of Substrate C to Substrate B may be about 3:1, about 2.5:1, about 2:1, about 1.5:1, or about 1:1. In examples, the ratio of Substrate C to Substrate A may be about 1.75:1, about 1.5:1, about 1.25:1, about 1:1, or about 0.75:1. In examples, the ratio of Substrate A:Substrate B may be about 4.25:1, about 4:1, about 3.75:1, about 3.5:1, about 3.25:1, about 3:1, or about 2.75:1.

The multidifferential agar 100 may include an inhibitor of gram-positive bacteria. Gram-positive bacteria have a thick peptidoglycan wall surrounding a cell membrane, whereas gram negative bacteria have a thinner peptidoglycan wall that is surrounded by an inner membrane and an outer membrane. Examples of molecules that inhibit gram-positive bacteria include bile salts, crystal violet and gram-positive antibiotics. Bile salts may be used as an inhibitor of gram-positive bacteria in the multidifferential agar 100 at a concentration of, for example, between about 0.05% (w/v) and about 0.5% (w/v) mg/L or between about 0.1% (w/v) and about 0.5% (w/v). Examples of gram-positive antibiotics include vancomycin, penicillin, cloxacillin, and erythromycin. Vancomycin may be used as an inhibitor of gram-positive bacteria in the multidifferential agar at a concentration of, for example, between about 0.00001% (w/v) and about 0.001% (w/v), or between about 0.0001% (w/v) and about 0.001% (w/v). In examples, the multidifferential agar 100 may include at least two inhibitors of gram-positive bacteria, such as bile salts and a gram-positive antibiotic.

The multidifferential agar 100 may include an antimycotic. Antimycotic may refer to a substance that inhibits the growth of or kills fungus. Examples of antimycotics include: polyene antifungals; imidazole, triazole, and thiazole antifungals; amorolfine; butenafine; naftifine; and terbinafine; anidulafungin; caspofungin; micafungin; balsam of Peru; ciclopirox; and orotomide. Examples of polyene antifungals include: amphotericin b; candicidin; filipin; hamycin; natamycin; and nystatin. Examples of imidazole antifungals include: clotrimazole; econazole; miconazole; and ketoconazole. Examples of triazole antifungals include: fluconazole; itraconazole; posaconazole; and voriconazole. An example of a thiazole antifungals is abafungin. Amphotericin b may be used in the multidifferential agar 100 at a concentration of between about 0.00001% (w/v) and about 0.001% (w/v), or between about 0.0001% (w/v) and about 0.001% (w/v).

The multidifferential agar 100 may include propylene glycol. Propylene glycol may be used in the multidifferential agar to provide a secondary level of differentiation between Salmonella and other organisms present on the plate. When Salmonella is present on a multidifferential agar, propylene glycol in the multidifferential agar 100 may cause a light-colored or white ring or halo to form around the Salmonella colonies resulting in “bullseye” colonies. In examples, the propylene glycol may be present in the multidifferential agar 100 at a concentration of between about 0.0001% and about 10%, or between about 0.001% and about 0.1%

The multidifferential agar 100 may include a base component. A base component may refer to the components of the multidifferential agar 100 that provide a solid matrix on which bacteria may grow (e.g., agar and/or gelatin), and provide nutrients to support bacterial growth, as well as additive such as buffers and/or emulsifiers. A solid matrix for bacterial growth may be a gelling agent that is nontoxic to the bacteria of interest (e.g., BTGN organisms, E. coli, other coliforms, and Salmonella), and is resistant to the metabolism of the bacteria to be cultured. Examples of gelling agents that may be used in a base component include agar, agarose, carrageenan, guar gum, gellan gum, isubgol, and/or gelatin. Nutrients present in a base component of a multidifferential agar 100 can include, for example, amino acids, vitamins, carbohydrates, nitrogen, and salts. Nutrients in a base component of a multidifferential agar 100 may be in the form of, for example, a digested protein (e.g., tryptone and/or peptone) and/or a meat or yeast extract. Nutrients may be provided in the multidifferential agar 100 as a bacterial growth medium. Examples of nutrients for bacterial growth include beef extract, gelatin, lactose, tryptone, L-cystine, and yeast extract. Beef extract may be present in the multidifferential agar 100 at a concentration between about 0.5 g/L and about 50 g/L, or between about 1 g/L and 10 g/L. Gelatin may be present in the multidifferential agar 100 at a concentration between about 0.01 g/L and about 50 g/L, or between about 0.1 g/L and 10 g/L. Lactose may be present in the multidifferential agar 100 at a concentration between about 0.1 g/L and about 100 g/L, or between about 1 g/L and about 50 g/L. Tryptone may be present in the multidifferential agar 100 at a concentration between about 0.1 g/L and about 100 g/L, or between about 1 g/L and about 20 g/L. Examples of other additives that may be present in a base component include surfactant such as PEG or Polysorbate 80 and/or buffers such as tris(hydroxymethyl)aminomethane (TRIS) and/or trisodium citrate, which also acts an emulsifier. Polysorbate 80 may be present in the multidifferential agar 100 at a concentration of between about 0.1 mL/L and about 100 mL/L, or between about 1 mL/L and about 20 mL/L. TRIS may be present in the multidifferential agar at a concentration of between about 0.01 g/L and about 50 g/L or between about 0.1 g/L and about 5 g/L. Trisodium citrate present in the multidifferential agar at a concentration of between about 0.01 g/L and about 25 g/L or between about 0.1 g/L and about 5 g/L.

The base component in the multidifferential agar 100 may include agar. Agar is a mix of the polysaccharide agarose, and a heterogeneous mixture of smaller molecules called agaropectin. The agar in the multidifferential agar 100 may be present at a concentration of, for example, between about 0.1% (w/v) and about 10% (w/v) or between about 0.5% (w/v) and about 5% (w/v).

The base component in the multidifferential agar 100 may include selective agents such as bile salts or acids, which are also surfactants. Bile salts may refer to the sodium and potassium salts of bile acids that are conjugated with taurine or glycine in the liver. Bile acids are steroid acids found predominantly in the bile of mammals and other vertebrates. Certain bacteria, such as members of the families Enterobactericeae, Pseudomonaceae, and Aeromonaceae are known as Bile-Tolerant Gram-negative organisms (including E. coli, other coliforms, and Salmonella) which are not inhibited by the presence of bile salts in growth media. In examples, bile salts present in the multidifferential agar may be provided as sodium deoxycholate, oxbile sodium cholate, or a mixture thereof. Selective agents can be included at a concentration of between about 0.001% (w/v) and about 5% (w/v) or between about 0.01% (w/v) and about 2% (w/v).

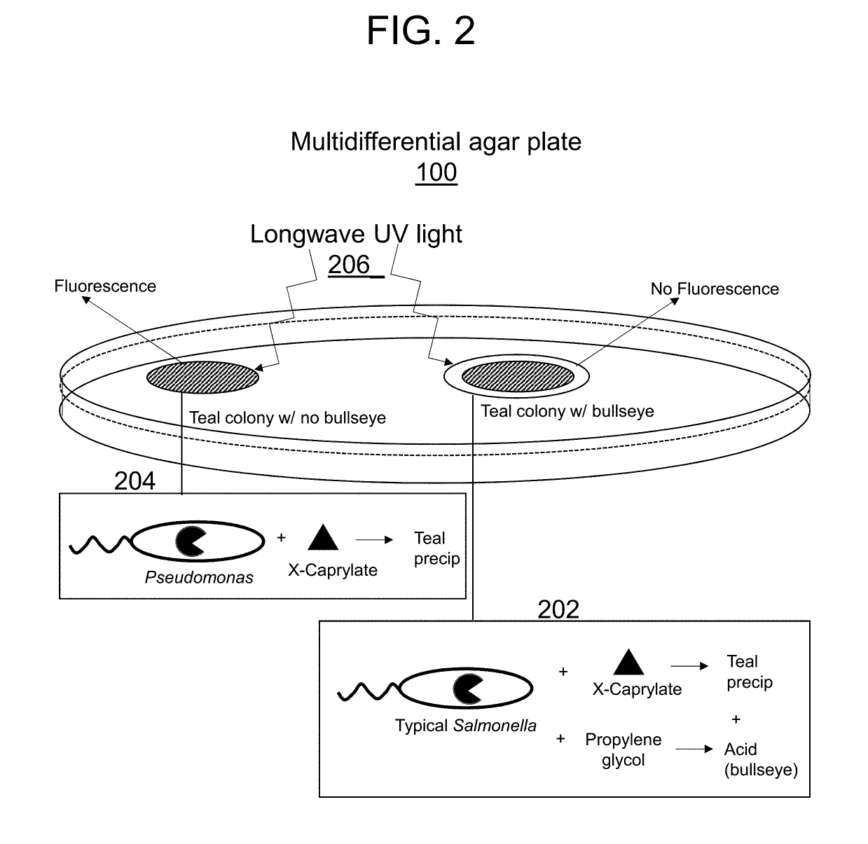

FIG. 2 illustrates an example of using a multidifferential agar 100 for detecting and differentiating between Salmonella and Pseudomonas.

A colony of Salmonella 202 on a multidifferential agar 100 may include a precipitate due to the cleavage of a chromogenic substrate for C-8 esterase (also referred to as Substrate C). In the examples shown in FIGS. 1 and 2, X-Caprylate is used as Substrate C, which may cause the colony to appear aqua or teal. Furthermore, the colonies may be surrounded by a white or lighter-colored ring or halo in a bullseye appearance, due to the presence of propylene glycol.

X-Caprylate may refer to the molecule 5-Bromo-4-chloro-3-indoxyl caprylate. When X-Caprylate is cleaved may yield a blue precipitate (Aₘₐₓ=615 nm). In examples, Substrate C (e.g., X-Caprylate) may be present in the multidifferential agar 100 at a concentration of between about 0.001% (w/v) and about 0.4% (w/v) or between about 0.017% (w/v) and about 0.032% (w/v), or between about 0.012% and 0.018% (w/v), or about 0.01% (w/v), about 0.015% (w/v), about 0.016% (w/v), about 0.02% (w/v), or about 0.032% (w/v). Atypical species of Salmonella (e.g., Salmonella arizonae) may express β-galactosidase as well as C8 esterase and therefore include a pink precipitate and a teal precipitate, which may result in blue-purple colonies with the unique bullseye ring appearance. The bullseye is only demonstrated on multidifferential agar 100 by Salmonella species. Therefore, colonies of typical Salmonella species (e.g., Salmonella typhimurium) on a multidifferential agar plate 100 may include a single precipitate (from Substrate C), and colonies of atypical Salmonella species may include two precipitates (from Substrate A and Substrate C), but both typical and atypical Salmonella species include a white ring/bullseye. The bullseye may be particularly useful for differentiating between typical Salmonella and Pseudomonas, because both react with Substrate C but not Substrate A, and therefore the resulting colonies may be a similar color.

The white ring surrounding a colony of Salmonella 202 on a multidifferential agar plate 100, may be due to the Salmonella metabolizing propylene glycol, leading to acidification of the environment immediately surrounding the colony and the appearance of a white ring or bullseye around the colony. The white ring surrounding a typical Salmonella species may appear white, white-ish blue, or lighter blue than the colony. The propylene glycol may be present in the multidifferential agar 100 at a concentration of between about 0.0001 and about 2%, or between about 0.001% and about 0.1%.

A colony of Salmonella 202 on a multidifferential agar 100 may not fluoresce in the presence of longwave ultraviolet (UV) light. A UV lamp or flashlight may be placed over the plate and the colonies observed to detect whether the colony fluoresces.

A colony of Pseudomonas 204 on a multidifferential agar 100 may include a precipitate formed by the hydrolysis of Substrate C. When Substrate C is X-Caprylate, the precipitate may be a teal precipitate, which may cause the colony to appear teal, whereas the organisms are inherently yellow to green and may lack a white or lighter-colored ring or halo.

The teal precipitate within a colony of Pseudomonas 204 may be due to cleavage of Substrate C (e.g., X-Caprylate) by C8 esterase, which is expressed by Pseudomonas. X-Caprylate may refer to the molecule 5-Bromo-4-chloro-3-indoxyl caprylate. When X-Caprylate is cleaved, it and may yield a teal precipitate (Aₘₐₓ=615 nm). In examples, Substrate C (e.g., X-Caprylate) may be present in the multidifferential agar 100 at a concentration of between about 0.001% (w/v) and about 0.4% (w/v), or between about 0.01% (w/v) and about 0.02% (w/v), or between about 0.012% and 0.018% (w/v), or about 0.01% (w/v), about 0.015% (w/v) about 0.02% (w/v), or about 0.016% (w/v.

On a multidifferential agar plate 100, a colony of Pseudomonas 204 may not possess a white or lighter-colored ring because Pseudomonas may not metabolize propylene glycol to form an acid in the immediate environment surrounding the colony. The propylene glycol may be present in the multidifferential agar 100 at a concentration of between about 0.0001% and about 1%, or between about 0.001% and about 0.1%.

A colony of Pseudomonas 204 on a multidifferential agar 100 may fluoresce in the presence of longwave ultraviolet (UV) light. Upon removing the plate from the incubator, a UV lamp or flashlight may be placed over the plate and the colonies observed to detect whether the colony fluoresces. Therefore, observing colonies that include a teal precipitate in the presence of UV light may be useful for identifying Pseudomonas on a multidifferential agar.

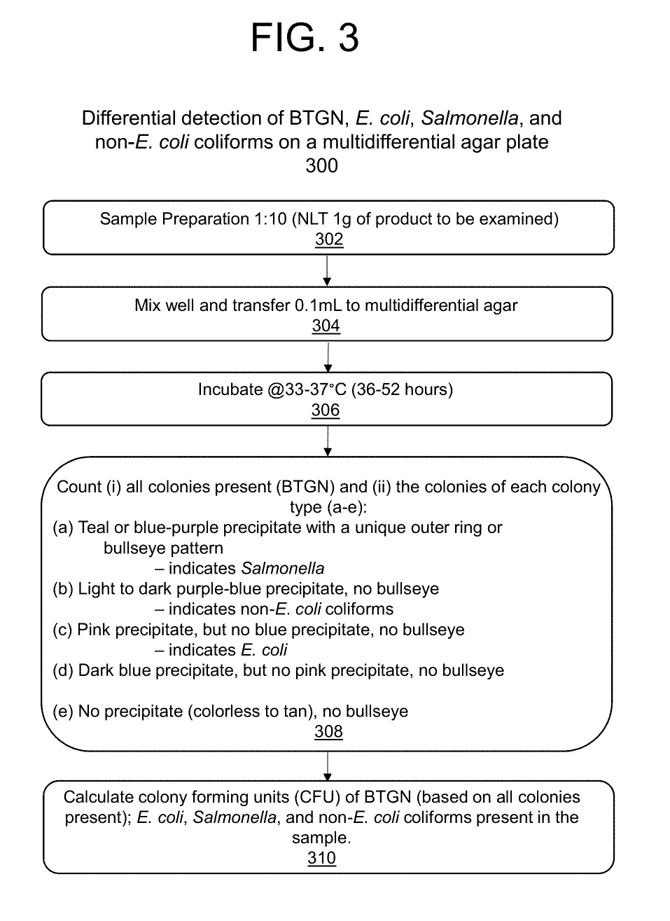

FIG. 3 illustrates a flow diagram of an example process 300 for differentially detecting Bile-Tolerant Gram-Negative organisms, E. coli, Salmonella, and non-E. coli coliforms on a multidifferential agar plate 300.

At block 302, the process 300 may include preparing a sample in a 1:10 dilution, using no less than 1 gram of the product to be examined. The sample may be dissolved or suspended in a sterile diluent, such as water or phosphate-buffered saline. For example, if the sample is a botanical product, the botanical product may be ground to a fine powder and suspended in the diluent. Some portion of, or all of the diluted/dissolved/suspended sample may be spread or otherwise placed onto the multidifferential agar plate using aseptic technique. For example, 0.1 mL of a sample suspended in a total of 1 mL deionized water may be placed onto the multidifferential agar plate using a sterile pipette.

At block 304, the process 300 may include mixing the sample well, and transferring an appropriate volume of the sample (e.g., 0.1 mL) to a multidifferential agar. The multidifferential agar may be present in a petri dish, such as a 10 cm petri dish. The volume of sample transferred may vary depending on the size of the container that holds the multidifferential agar.

At block 306, the process 300 may include incubating the multidifferential agar, typically between 33° C. and 37° C., for an appropriate duration of time (e.g., 36 to 52 hours). The incubation may be at a temperature of between 30° C. and 45° C., or between 33° C. and 37° C. Incubating the multidifferential agar plate may be performed for a period of time sufficient for colonies to form on the plate. The incubation may be performed for at least 24 hours, at least 36 hours, at least 48 hours, at least 60 hours, at least 72 hours, or at least 80 hours. The incubation may be performed with the plate inverted (i.e. upside-down) in an incubator.

At block 308, the process 300 may include determining the presence of (i) all colonies present (representative of BTGN); and (ii) the colonies of colony types with: (a) teal or blue-purple precipitate, with a unique outer ring or bullseye pattern, which do not fluoresce under longwave UV light; (b) light to dark purple-blue precipitate with no bullseye; (c) pink precipitate but no blue precipitate and no bullseye; (d) dark blue precipitate but no pink precipitate and no bullseye; and/or (e) colorless to tan colonies with no bullseye. The colonies may be inspected to determine the presence of the colony types by, for example, visual inspection, microscopy, and/or by spectrophotometry. Determining the presence of colony type (a) may be indicative of Salmonella. The colonies of colony type (a) may be any shade of blue or blue-green, such as, for example, light-blue, aqua, teal, or cyan or with atypical strains a blue-purple. While typical and atypical Salmonella may produce different colony colors on the multidifferential agar, both types produce the unique bullseye pattern, which will be present only with Salmonella species. Determining the presence of colony type (b) may be indicative of non-E. coli coliforms. The light to dark purple-blue colonies without a bullseye may be any shade of light to dark purple or violet-blue, such as, for example, lavender, fuchsia, or amethyst. Determining the presence of colony type (c) may be indicative of E. coli. The pink colonies without a bullseye may be any shade of pink, orange, orange-pink, or pink-red such as, for example, salmon, light pink, light orange, tangerine, or magenta. Determining the presence of colony type (d) may be indicative of BTGN organisms that express ß-D-Glucosidase, but not β-galactosidase nor C8-esterase. The colonies with dark blue precipitate, no pink precipitate, and no bullseye may be dark blue or black. Determining the presence of colony type (e) may be indicative of other BTGN organisms. Several BTGN organisms will grow on a multidifferential agar 100 with no response to any of the chromogenic substrates present. These organisms will grow as colorless, beige, or tan colonies and may include Proteus mirabilis and/or Shigella flexneri. These organisms should be included in the total count (CFU) for Bile-Tolerant Gram-Negative organisms. Additionally, several organisms will hydrolyze Substrate B resulting in blue-black colonies, such as Proteus vulgaris. These organisms should also be included in the total count (CFU) for BTGN organisms. (Proteus organisms can be further identified by swarming at room temperature.)

The results obtained by assaying the colonies may be compared to a positive control and/or a negative control. For example, a reference standard of Salmonella (e.g., S. typhimurium—ATCC® 14208) incubated on a multidifferential agar plate may be used as a positive control and the sterile dilute utilized for serial dilutions incubated on a multidifferential plate may be used as a negative control. As another example, a reference standard of a gram-positive bacteria, such as Staphyloccoccus (e.g, S. aureus—ATCC® 6583) incubated on a multidifferential agar plate may be used as a negative control.

At block 310, the process 300 may include calculating the colony-forming units (CFU) of BTGN organisms, E. coli, Salmonella, and non-E. coli coliforms present in the sample, respectively. The total colonies (including all colonies types) present on the multidifferential agar represents the total BTGN colonies on the multidifferential agar plate. CFU may be measured based on the number of viable bacteria that resulted in visible colonies on the multidifferential agar plate. For example, if ten colonies of colony type (c) are identified on the plate, and one-tenth of the total sample was spread onto the plate, then this would result in a calculation of 100 CFUs of E. coli in the sample.

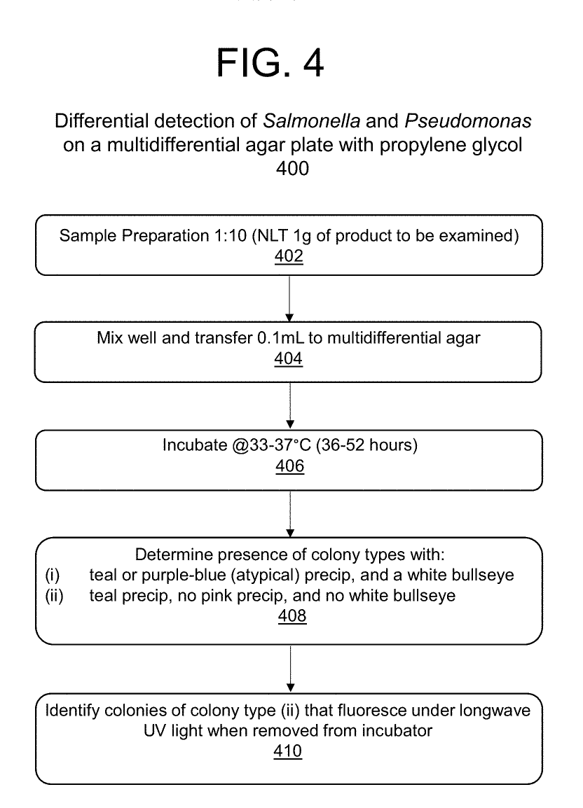

FIG. 4 illustrates a flow diagram of an example process 400 for differentially detecting Salmonella, and/or Pseudomonas on a multidifferential agar plate.

At block 402, the process 400 may include preparing a sample in a 1:10 dilution, using no less than 1 gram of the product to be examined. The sample may be dissolved or suspended in a sterile diluent, such as water or phosphate-buffered saline. For example, a product may be ground to a fine powder and suspended in the diluent. Some portion of, or all of the diluted/dissolved/suspended sample may be spread onto the multidifferential agar plate using aseptic technique. For example, 0.1 mL of a sample suspending in a total of 1 mL sterile deionized water may be spread onto the multidifferential agar plate using a sterile pipette and spreader.

At block 404, the process 400 may include mixing the sample well, and transferring an appropriate volume of the sample (e.g., 0.1 mL) to multidifferential agar. The multidifferential agar may be present in a petri dish, such as a 10 cm petri dish. The volume of sample transferred may vary depending on the size of the container that holds the multidifferential agar.

At block 406, the process 400 may include incubating the multidifferential agar, typically at between 33° C. and 37° C., for an appropriate duration of time (e.g., for 36 to 52 hours). The incubation may be at a temperature of between 30° C. and 45° C., or between 33° C. and 37° C. The incubation may be performed for 24 hours, at least 36 hours, at least 48 hours, at least 60 hours, at least 72 hours, or at least 80 hours. The incubation may be performed with the plate inverted (i.e. upside-down) in an incubator.

At block 408, the process 400 may include determining the presence of colony types with: (i) teal or purple-blue (atypical) precipitate, and a white ring or bullseye appearance; and (ii) teal precipitate, no pink precipitate, and no white ring or bullseye. Determining the presence of colony types (i) and (ii) may include visually inspecting the plate to identify aqua or teal colonies. The aqua or teal colonies may be any shade of blue or blue-green, such as, for example, light-blue, aqua, teal, or cyan. Determining the presence of a white ring or bullseye pattern may include visually inspecting the colony to determine if a white or lighter colored ring or halo surrounds the colony or examining the plate over white or black backgrounds. The ring or halo may be, for example 0.5 mm to 2 mm wide surrounding the aqua or teal colony. If the multidifferential agar plate contains colonies with teal precipitate, no pink precipitate, and a white ring/bullseye, this may indicate the presence of typical Salmonella (e.g., S. typhimurium). In contrast to typical Salmonella, atypical Salmonella species may include a teal precipitate and a pink precipitate (causing them to appear blue-purple), as well as a bullseye. The unique bullseye pattern will be present only with Salmonella species (including typical and atypical Salmonella).

At block 410, the process 400 may include identifying colonies of colony type (ii) with teal precipitate, no pink precipitate, and no white ring, which fluoresce under UV light when removed from the incubator. If colonies are identified that (a) include: teal precipitate, no pink precipitate, and no white ring, and (b) fluoresce under longwave UV light, this may indicate the presence of Pseudomonas. Many species of Pseudomonas (e.g., Pseudomonas aeruginosa) fluoresce under long wave UV whereas Salmonella will not.

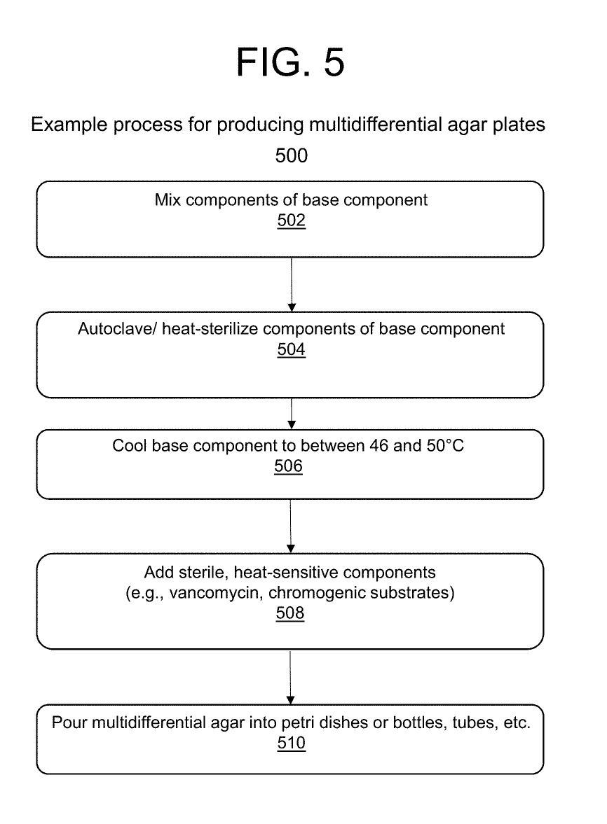

FIG. 5 illustrates a process 500 for producing multidifferential agar plates.

At block 502, the process 500 may include mixing the components of a base component. The base component may include: gelatin (e.g., about 2 g/L to about 6 g/L, or about 3 g/L to about 5 g/L, or about 4 g/L), tryptone (e.g., about 2 g/L to about 6 g/L, or about 3 g/L to about 5 g/L, or about 4 g/L), beef extract (e.g., about 0.5 g/L to about 10 g/L, or about 1 g/L to about 5 g/L or about 3 g/L), lactose (e.g., about 1 g/L to about 30 g/L, or about 10 g/L to about 20 g/L or about 15 g/L), agar (e.g., about 1 g/L to about 30 g/L, or about 10 g/L to about 20 g/L or about 15 g/L), Polysorbate 80 (e.g., about 0.5 mL/L to about 15 mL/L, or about 1 mL/L to about 10 mL/L, or about 5 mL/L), propylene glycol (e.g., about 1 mL/L to about 20 mL/L, or about 5 mL/L to about 15 mL/L, or about 10 mL/L), TRIS (e.g., about 0.1 g/L to about 1 g/L, or about 0.2 g/L to about 0.8 g/L, or about 0.60 g/L), sodium citrate (e.g., about 0.1 g/L to about 1 g/L, or about 0.2 g/L to about 0.8 g/L, or about 0.50 g/L), sodium deoxycholate (e.g., about 0.25 g/L to about 25 g/L, or about 1 g/L to about 5 g/L, or about 2.5 g/L), oxbile (e.g., about 0.1 g/L to about 10 g/L, or about 0.5 g/L to about 5 g/L, or about 1.0 g/L), and L-cystine (e.g., about 0.01 g/L to about 0.5 g/L, or about 0.1 g/L to about 0.2 g/L, or about 0.128 g/L). The components of the base component may be dissolved or suspended in water or other suitable solvents.

At block 504, the base component may be autoclaved/heat sterilized. After autoclaving, the process 500 may include cooling the base agar to between about 46° C. and about 50° C. 506. The cooling operation may be performed, for example, to allow heat-sensitive components to be added before the agar solidifies.

At block 508, the process 500 may include adding heat-sensitive components (e.g., vancomycin, amphotericin b, and chromogenic substrates). The heat-sensitive components may be sterilized prior to adding the components, such as by sterile filtering. For example, a C8-esterase chromogenic substrate such as X-Caprylate (e.g., about 0.01 g/L to about 40 g/L, or about 0.1 g/L to about 4.0 g/L, or about 0.15 g/L to about 0.35 g/L, or about 0.25 g/L), a β-galactosidase chromogenic substrate such as Salmon-Gal (e.g., about 0.01 g/L to about 1 g/L, or about 0.15 g/1 to about 0.30 g/L, or about 0.1 g/L to about 1.0 g/L, or about 0.20 g/L), and a ß-D-Glucosidase chromogenic substrate such as X-Glucoside (e.g., about 0.01 g/L to about 1 g/L, or about 0.03 g/L to about 0.10 g/L, or about 0.05 g/L to about 1.0 g/L, or about 0.08 g/L), vancomycin (e.g., about 0.4 mg/L to about 40 mg/L, or about 1 mg/L to about 10 mg/L, or about 4 mg/L), and/or amphotericin B (e.g., about 0.6 mg/L to about 60 mg/L, or about 1 mg/L to about 10 mg/L, or about 6 mg/L) may be sterile filtered into the base agar and mixed thoroughly to produce a multidifferential agar.

At block 510, the process 500 may include pouring the multidifferential agar into petri dishes or plates. The petri dishes or plates may include for, example, any object that the multidifferential agar can be applied to form a flat surface to support bacterial growth. For example, the multidifferential agar may be applied to a thin film, upon which a sample may be plated for bacterial detection or filled into tubes or bottles, with or without the presence of agar.

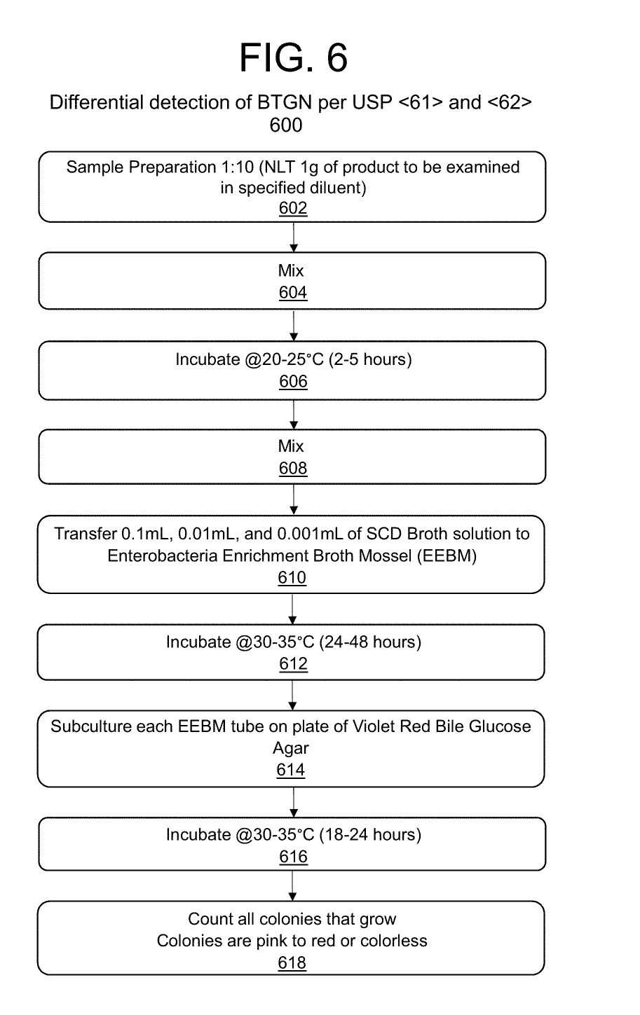

FIG. 6 illustrates a workflow 600 that can be used to identify bile-tolerant gram-negative (BTGN), using techniques outlined in United States Pharmacopeia (USP) General Chapters <61> and <62>. The workflow 600 can include, at block 602, sample preparation at a ratio of 1:10 (e.g., no less than lg of product to be examined in a specified diluent). The specified diluent may be soybean-casein digest (SCD) broth, for example. The workflow 600 may also include, at block 604, mixing the sample. Examples of methods to mix the sample include, for example, pipetting or gently vortexing. The workflow 600 may include, at block 606, incubating the sample at 20-25° C. for two to five hours. After incubating the sample, the sample may be mixed at block 608. Next, the workflow 600 may include, at block 610, transferring 0.1 mL, 0.01 mL, and 0.001 mL of SCD Broth solution to Enterobacteria Enrichment Broth Mossel (EEBM). Once the sample is in the EEBM broth, the workflow 600 may include, at block 612, incubating the sample at 30-35° C. for twenty-four to forty-eight hours.

After incubating, the workflow 600 may next include, at block 614, subculturing each of the EEBM tubes on a plate of Violet Red Bile Glucose (VRBG) agar. Next, the workflow 600 may include, at block 616, incubating the VRBG agar plates at 30-35° C. for eighteen to twenty-four hours.

After the final incubating step, the workflow 600 may next include, at block 618, counting colonies that grow. The colonies may be pink to red, or colorless. Any growth on the plates indicates a positive result for the presence of BTGN organisms. The most probable number of BTGN in the original sample can be determined by counting the colonies on each of the different dilution plates and estimating the most probable number (MPN) of BTGN present using a microbiology technique referred to as the MPN method. Control organisms that can be utilized when performing the workflow 600 include the compendial organisms: E. coli strain ATCC® 8739 (a positive control), Pseudomonas aeruginosa strain ATCC® 9027 (a positive control), and Staphylococcus aureus strain ATCC® 6538 (a gram-positive, negative control, which should be inhibited). USP <61> and <62> identifies the test strains to be utilized along with the ATCC®, NCIMB, CIP, or NBRC number for each strain. These are a standardized set of organisms utilized for growth promotion testing.

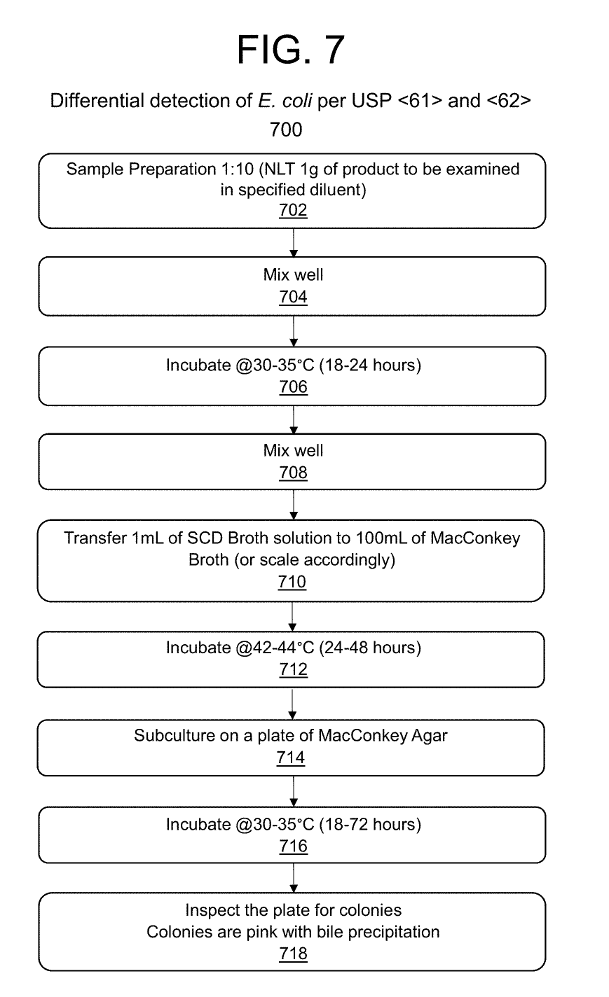

FIG. 7 illustrates a workflow 700 that can be used to identify E. coli using techniques outlined in United States Pharmacopeia (USP) General Chapters <61> and <62>. The workflow 700 can include, at block 702, sample preparation at a ratio of 1:10 (e.g., no less than lg of product to be examined in a specified diluent). The specified diluent may be soybean-casein digest (SCD) broth. The workflow 700 may include, at block 702, mixing the sample. Examples of methods to mix the sample include, for example, pipetting or gently vortexing. The workflow 700 may include, at block 706, incubating the sample at 30-35° C. for eighteen to twenty-four hours. After incubating the sample, the sample, at block 708, may be mixed. The workflow 700 may also include, at block 710, transferring 1mL of SCD broth solution to 100 mL of MacConkey broth. The volumes of SCD broth solution and MacConkey broth may be scaled accordingly. Once the sample is in the MacConkey broth, the workflow 700 may include, at block 712, incubating the sample at 40-42° C. for twenty-four to forty-eight hours.

After incubation, the workflow 700 may include, at block 714, subculturing the MacConkey broth solution on a MacConkey agar. The workflow 700 may also include, at block 716, incubating the MacConkey agar plate at 30-35° C. for eighteen to seventy-two hours.

After the final incubating step, the workflow 700 may include, at block 718, inspecting the plate for colonies. E. coli colonies will typically be pink with bile precipitation. However, any colony growth on the plates indicates the possible presence of E. coli. The product complies with the test if no colonies are present or identifications are negative for E. coli. Control organisms that can be utilized when performing the workflow 700 include the compendial organisms: E. coli strain ATCC® 8739 (a positive control), and Staphylococcus aureus strain ATCC® 6538 (a gram-positive, negative control, which should be inhibited).

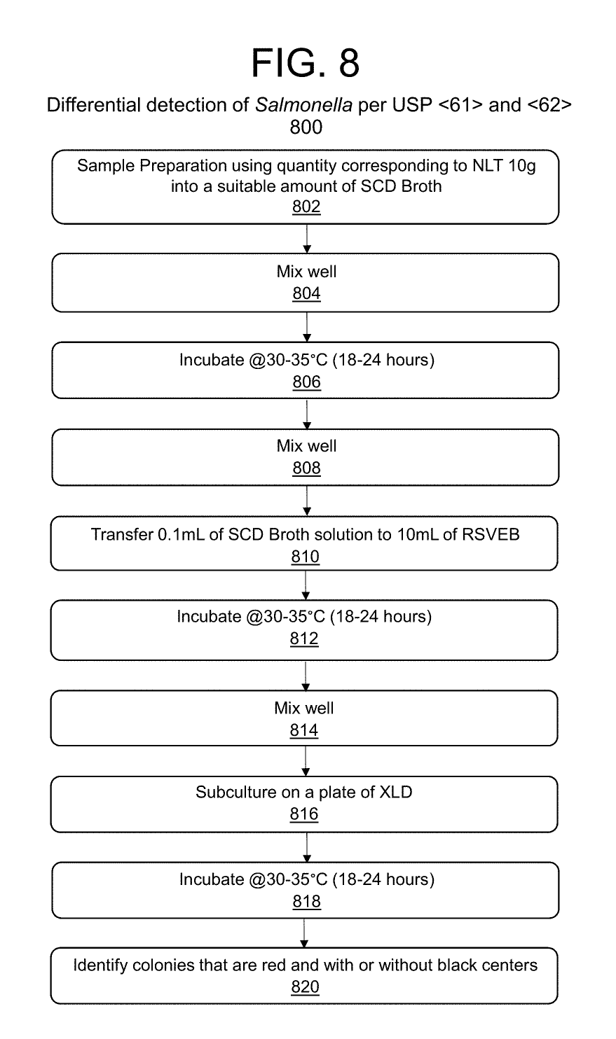

FIG. 8 illustrates a workflow 800 that can be used to identify Salmonella using techniques outlined in United States Pharmacopeia (USP) General Chapters <61> and <62>. The workflow 800, at block 802, can include sample preparation using a quantity corresponding to no less than 10 g into a suitable amount of SCD Broth. The amount of SCD broth may be determined as per the suitability of the test method. The specified diluent may be soybean-casein digest (SCD) broth. The workflow 800 may also include, at block 804, mixing the sample. Examples of methods to mix the sample include, for example, pipetting or gently vortexing. The workflow 800 may also include, at block 806, incubating the sample at 30-35° C. for eighteen to twenty-four hours. After incubating the sample, the sample may be mixed at block 808. The workflow 800 may also include, at block 810, transferring 0.1 mL of SCD broth solution to 10 mL of Rappaport Vassiliadis Salmonella Enrichment Broth (RVSEB). Once the sample is in the RSVEB, the workflow 800 may include, at block 812, incubating the sample at 30-35° C. for eighteen to twenty-four hours.

After incubating, the workflow 800 may include, at block 814, mixing the sample. The workflow 800 may also include, at block 816, subculturing the sample on a plate of xylose lysine deoxycholate agar (XLD). The XLD plate may be incubated, at block 818, at 30-35° C. for eighteen to twenty-four hours.

After the final incubating step, the workflow 800 may include, at block 820, identifying red colonies with or without black centers. The possible presence of Salmonella is indicated by well-developed, red colonies, with or without black centers. This can be confirmed with further identification tests. The tested product complies with the test if colonies of the types previously described are not present or identifications are negative for Salmonella. A control organism that can be utilized when performing the workflow 800 includes the compendial organism Salmonella typhimurium, strain ATCC® 14028 (a positive control).

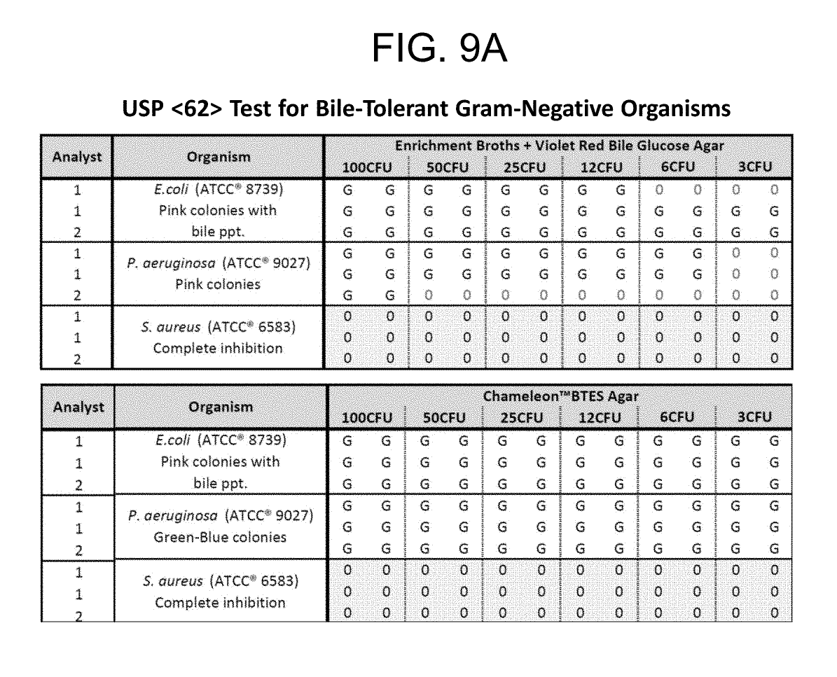

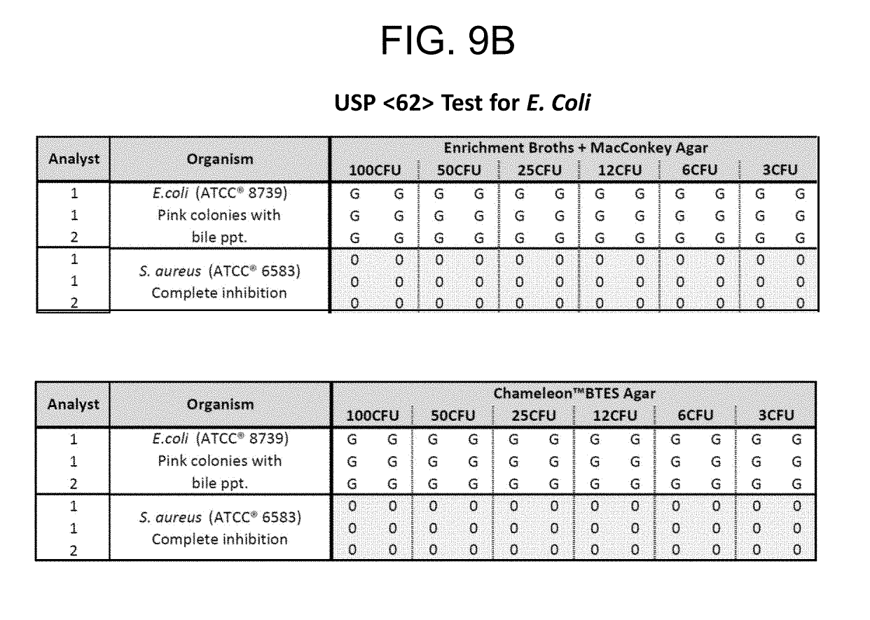

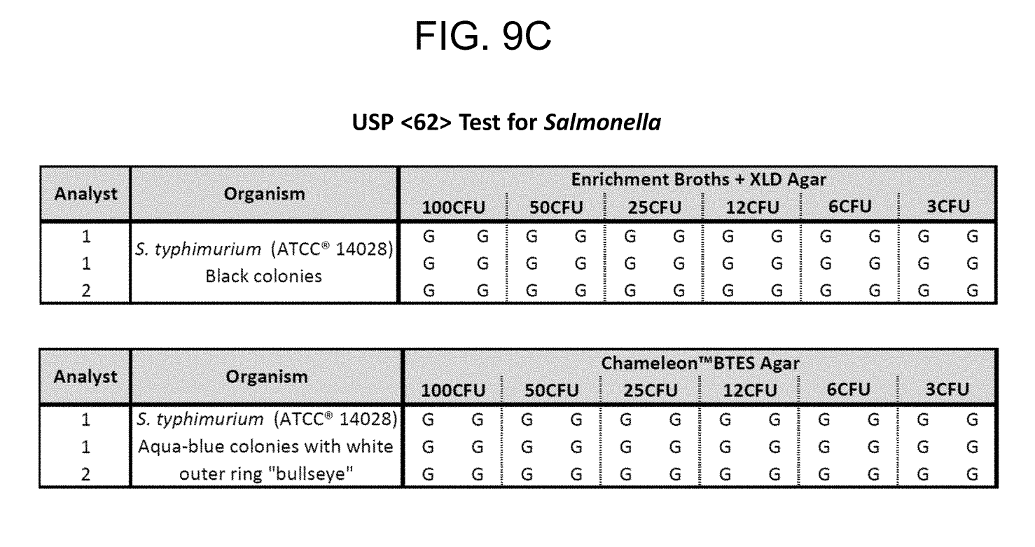

FIGS. 9A-9C illustrate results from a comparison study using a multidifferential agar versus the traditional medium outlined in United States Pharmacopeia (USP) General Chapters <61> and <62>, which specifically include the tests for E. coli, Salmonella, and BTGN organisms. FIG. 9A shows summary data for the equivalency study where recoveries for BTGN organisms between the compendial agars and the multidifferential agar were compared without performing enrichment steps or pre-incubations. A “G” represents detected growth of the organism, while “0” represents complete inhibition of growth of the organism. As can be seen in this comparison study, the presently described multidifferential agar provides for detection of organisms at CFUs that compendial agars do not. Specifically, E. coli was detected using the multidifferential agar described herein at 6 CFUs and 4 CFUs, while E. coli was not detected at these CFUs using the compendial agar. The same result is seen with respect to P. aeruginosa at 50 CFUs, 25 CFUs, 12 CFUs, 6 CFUs, and 3 CFUs.

FIG. 9B shows results of tests comparing compendial methods versus multidifferential agar methods for detecting and enumerating E. coli. FIG. 9B shows that the results of testing for E. coli utilizing the multidifferential agars and methods described herein work at least as well as the compendial methods.

FIG. 9C shows results of tests comparing compendial methods versus multidifferential agar methods for detecting and enumerating Salmonella. FIG. 9C shows that the results of testing for Salmonella utilizing the multidifferential agars and methods described herein work at least as well as the compendial methods.

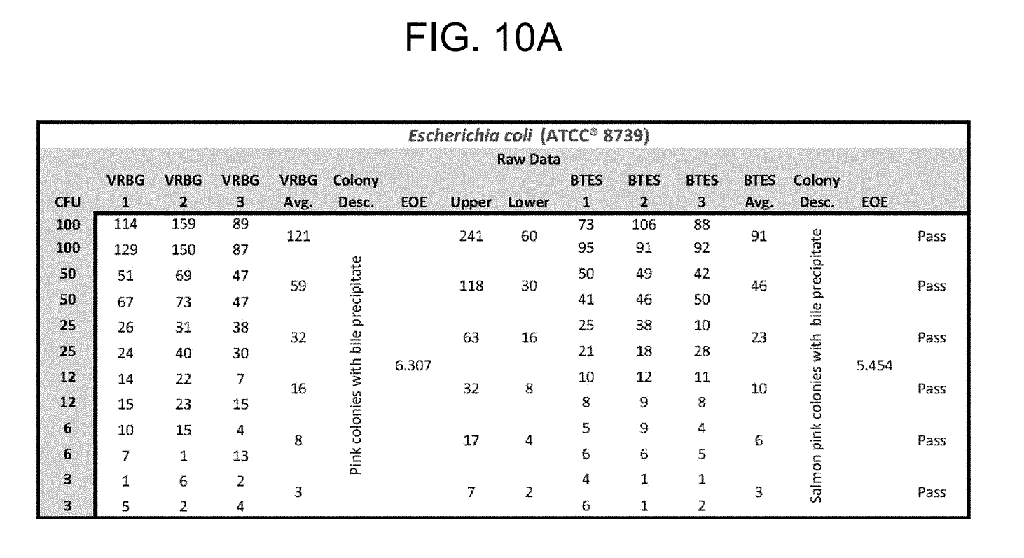

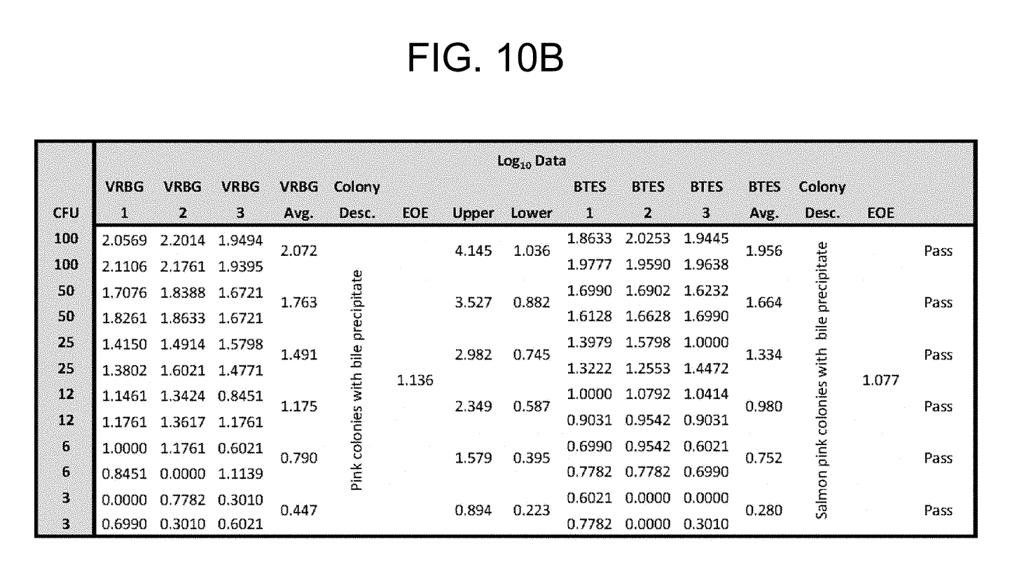

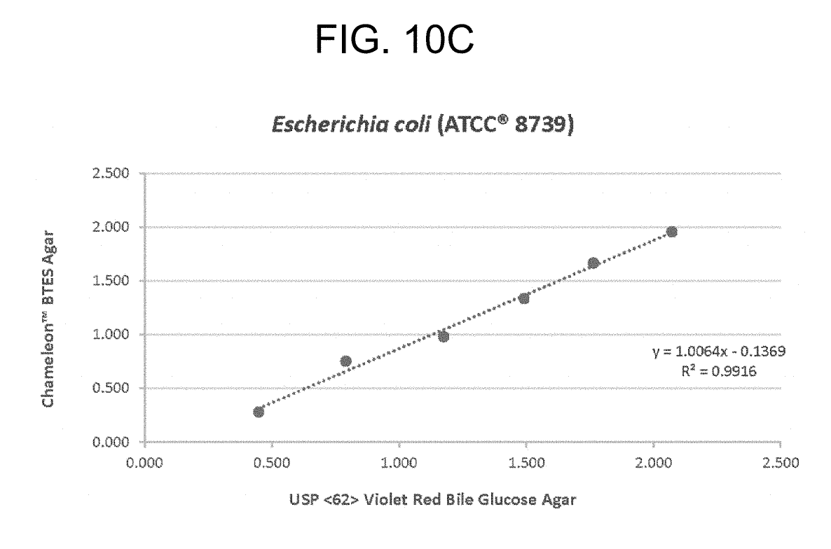

FIGS. 10A-C illustrate results for detecting and enumerating E. coli utilizing the multidifferential agars and methods described herein. Violet Red Bile Glucose compendial agar is shown in the table on the left-hand side, marked as “VRBG.” FIG. 10A shows that the multidifferential agar described herein provides for better E. coli detection results as compared to compendial agars. As shown in FIG. 10A, and elsewhere herein, “EOE” is defined as error of estimate. FIG. 10B shows Log₁₀ data for detecting and enumerating E. coli utilizing the multidifferential agars described herein and compendial agars. Again, FIG. 10B shows that the multidifferential agars described herein perform better than the compendial agars. FIG. 10C shows a chart illustrating the result comparison between detection results using the multidifferential agar described herein and the compendial agar. As seen from FIG. 10C, the error or difference between compendial agar use and the multidifferential agar described herein is minimal.

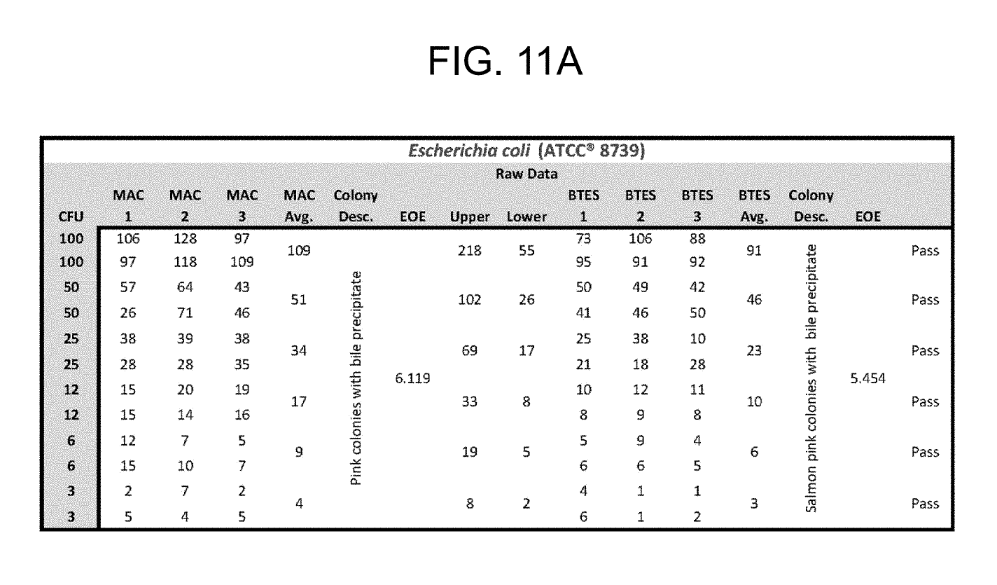

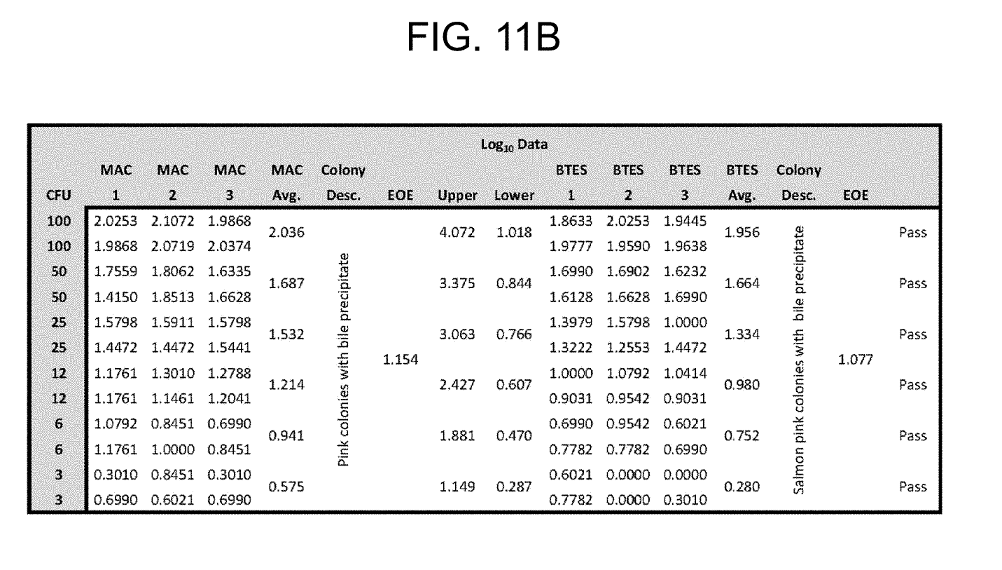

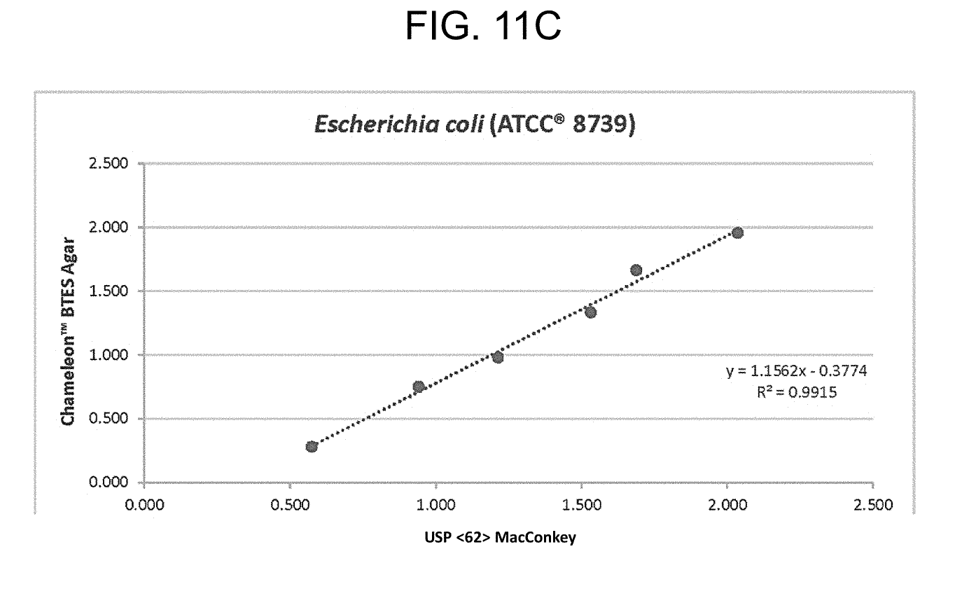

FIGS. 11A-C illustrate additional results for detecting and enumerating E. coli utilizing the multidifferential agars and methods described herein. FIGS. 11A-C show similar types of data as FIGS. 10A-C, described above, but are taken from a second comparison test. The results shown in FIGS. 11A-C bolster the results in FIGS. 10A-C and further illustrate that the multidifferential agar described herein provides for detection and enumeration of E. coli better than compendial agars.

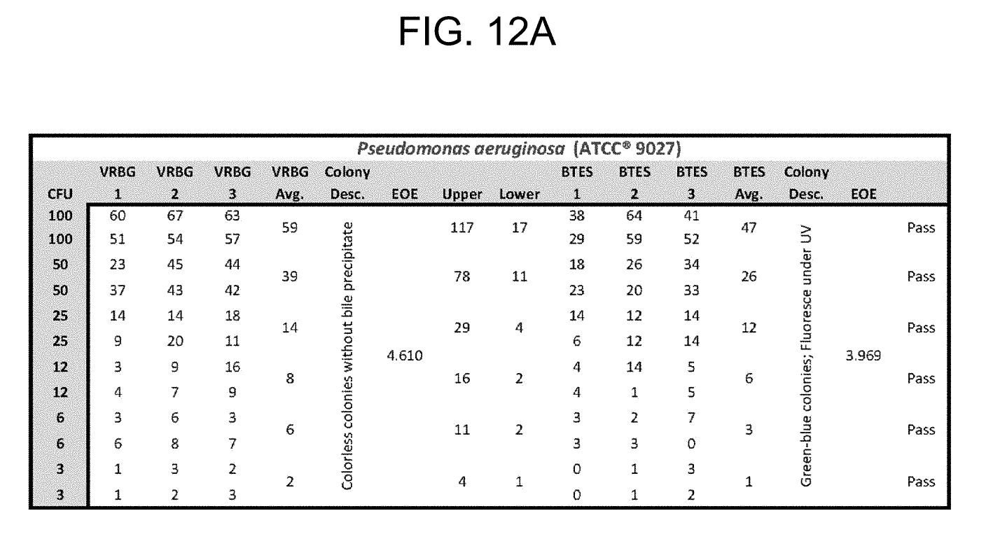

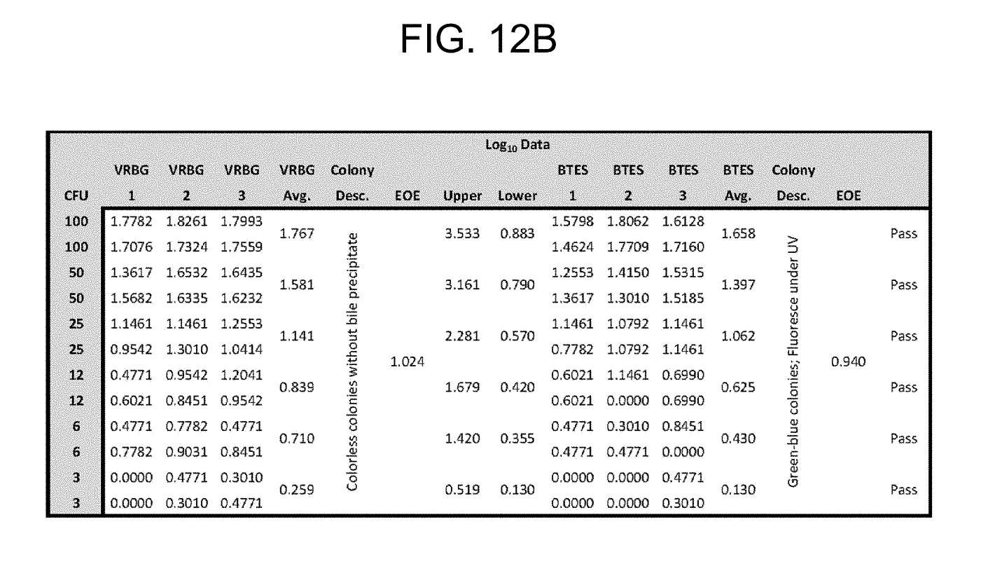

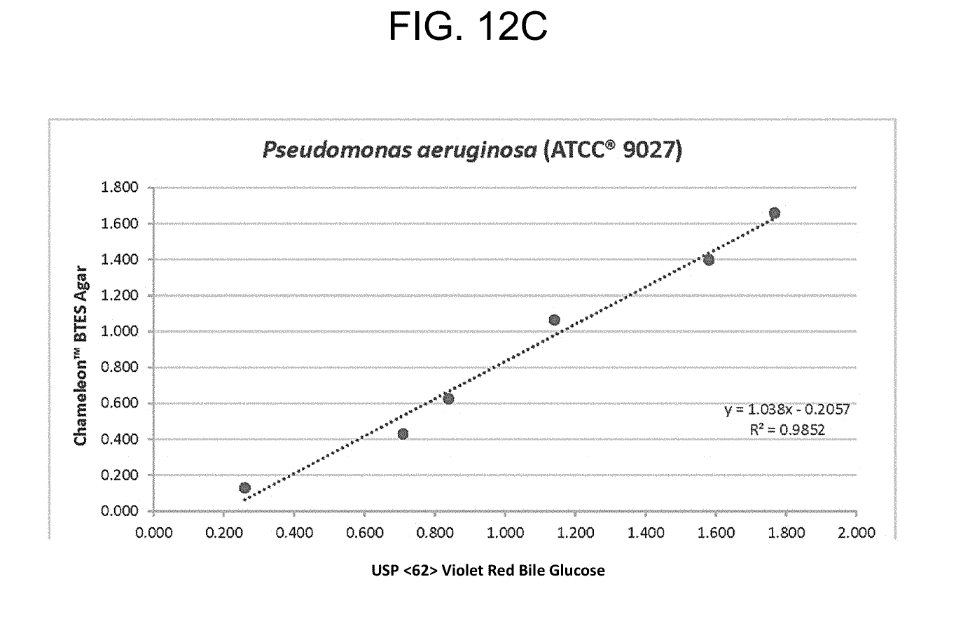

FIGS. 12A-C illustrate results for detecting and enumerating Pseudomonas aeruginosa utilizing the multidifferential agars and methods described herein. FIG. 12A shows that the multidifferential agar described herein provides for Pseudomonas aeruginosa detection results that are better than compendial agars, as evidenced by at least the error of estimate rating. FIG. 12B shows Log₁₀ data for detecting and enumerating Pseudomonas aeruginosa utilizing the multidifferential agars described herein and compendial agars. Again, FIG. 12B shows that the multidifferential agars described herein perform better than the compendial agars. FIG. 12C shows a chart illustrating the result comparison between detection results using the multidifferential agar described herein and the compendial agar. As seen from FIG. 12C, the error or difference between compendial agar use and the multidifferential agar described herein is minimal.

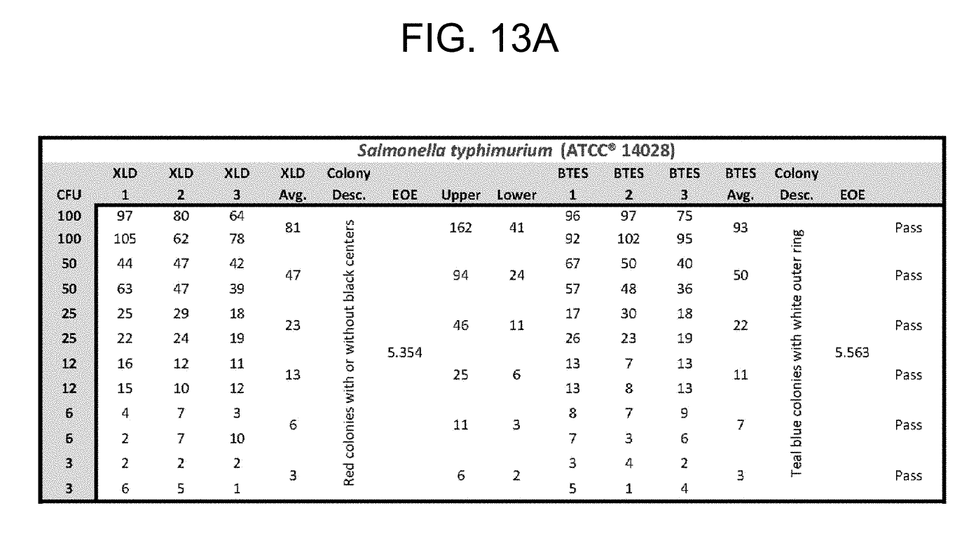

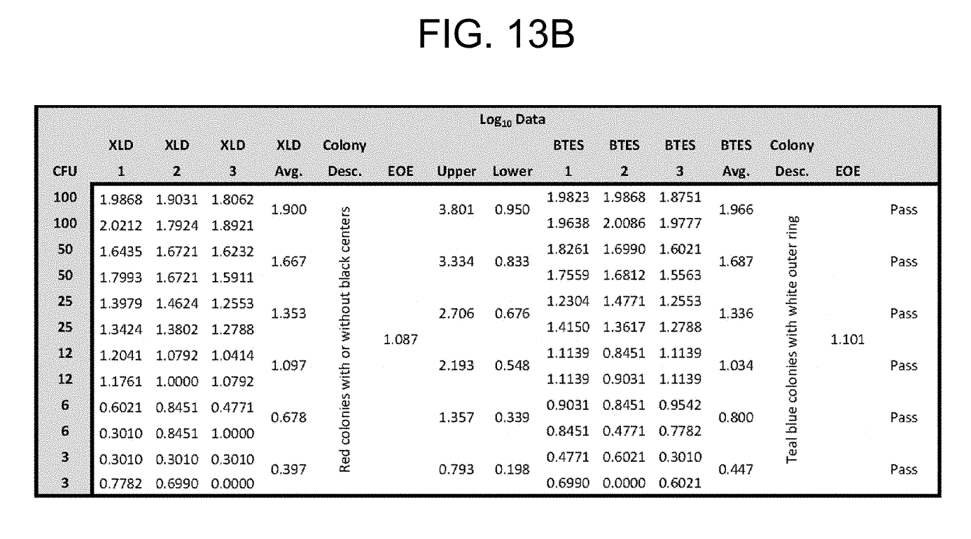

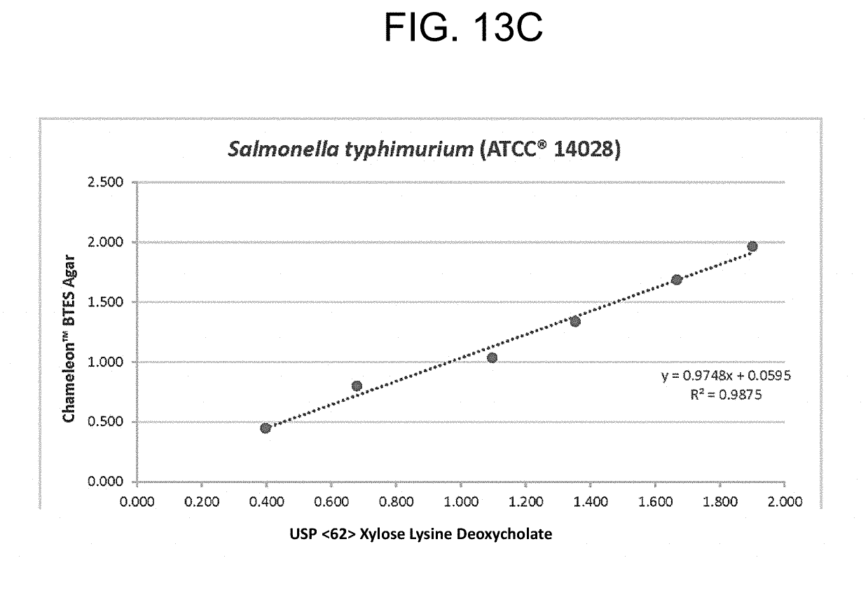

FIGS. 13A-C illustrate results for detecting and enumerating Salmonella typhimurium utilizing the multidifferential agars and methods described herein. FIG. 13A shows that the multidifferential agar described herein provides for Salmonella detection results that are better than compendial agars, as evidenced by at least the error of estimate rating. FIG. 13B shows Log₁₀ data for detecting and enumerating Salmonella utilizing the multidifferential agars described herein and compendial agars. Again, FIG. 13B shows that the multidifferential agars described herein perform similar to the compendial agars. FIG. 13C shows a chart illustrating the result comparison between detection results using the multidifferential agar described herein and the compendial agar. As seen from FIG. 13C, the error or difference between compendial agar use and the multidifferential agar described herein is minimal.

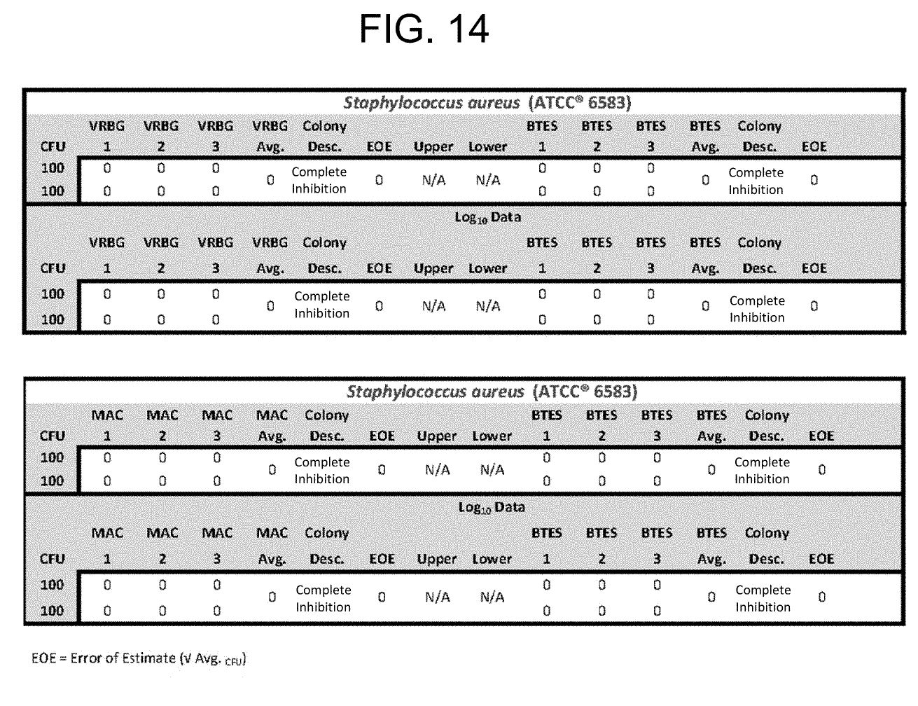

FIG. 14 illustrates inhibition of growth of Staphylococcus aureus on multidifferential agar and on the compendial agars Violet Red Bile Glucose and MacConkey. As shown in FIG. 14, the multidifferential agar inhibits growth of gram-positive bacteria.

The processes and operations described herein are illustrated as collections of blocks in logical flow diagrams, which represent a sequence of operations. Any number of the described blocks may be combined in any order and/or in parallel to implement the process, or alternative processes, and not all of the blocks need be executed. For discussion purposes, the processes are described with reference to the environments, architectures, and devices described in the examples herein, such as, for example those described with respect to FIGS. 1 and 2, although the processes may be implemented in a wide variety of other environments, architectures and devices.

While the foregoing invention is described with respect to the specific examples, it is to be understood that the scope of the invention is not limited to these specific examples. Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Although the application describes embodiments having specific structural features and/or methodological acts, it is to be understood that the claims are not necessarily limited to the specific features or acts described. Rather, the specific features and acts are merely illustrative of some embodiments that fall within the scope of the claims of the application.

What is claimed is:

1. A composition comprising:
(i) a base component comprising:
agar or broth solution;
a first inhibitor of gram-positive bacteria comprising bile salt;
propylene glycol;
a surfactant; and
a microbial growth medium;
(ii) a second inhibitor of gram-positive bacteria;
(iii) a C8-esterase chromogenic substrate, at a concentration of about 0.1 g/L to about 4.0 g/L;
(iv) a β-galactosidase chromogenic substrate, at a concentration of about 0.1 g/L to about 1.0 g/L; and
(v) a ß-D-Glucosidase chromogenic substrate, at a concentration of about 0.05 g/L to about 1.0 g/L.

2. The composition of claim 1, further comprising an antimycotic.

3. The composition of claim 1, wherein the C8-esterase chromogenic substrate comprises X-Caprylate.

4. The composition of claim 1, wherein the β-galactosidase chromogenic substrate comprises Salmon-Gal.

5. The composition of claim 1, wherein the ß-D-Glucosidase chromogenic substrate comprises X-Glucoside.

6. The composition of claim 1, wherein the C8-esterase chromogenic substrate is at a concentration of about 0.25 g/L.

7. The composition of claim 1, wherein the β-galactosidase chromogenic substrate is at a concentration of about 0.20 g/L.

8. The composition of claim 1, wherein the ß-D-Glucosidase chromogenic substrate is at a concentration of about 0.08 g/L.

9. A composition comprising:
(i) a base component comprising (a) agar or broth solution and (b) microbial growth medium; and
(ii) at least three chromogenic substrates comprising a C8-esterase chromogenic substrate, a β-galactosidase chromogenic substrate, and a ß-D-Glucosidase chromogenic substrate.

10. The composition of claim 9, wherein the base component further comprises bile salt.

11. The composition of claim 9, wherein the base component further comprises propylene glycol.

12. The composition of claim 9, wherein the C8-esterase chromogenic substrate comprises X-Caprylate.

13. The composition of claim 9, wherein the β-galactosidase chromogenic substrate comprises Salmon-Gal.

14. The differential agar of claim 9, wherein the ß-D-Glucosidase chromogenic substrate comprises X-Glucoside.

15. The differential agar of claim 9, wherein the C8-esterase chromogenic substrate is at a concentration of about 0.15 g/L to about 0.40 g/L.

16. The differential agar of claim 9, wherein the β-galactosidase chromogenic substrate is at a concentration of about 0.15 g/L to about 0.30 g/L.

17. The differential agar of claim 9, wherein the ß-D-Glucosidase chromogenic substrate is at a concentration of about 0.03 g/L to about 0.10 g/L.

18. A composition comprising:
agar or broth solution;
a microbial growth medium;
about 0.01% (w/v) to about 4.0% (w/v) of a C8-esterase chromogenic substrate;
about 0.015% (w/v) to about 0.030% (w/v) of a β-galactosidase chromogenic substrate; and
about 0.003% (w/v) to about 0.010% (w/v) of a ß-D-Glucosidase chromogenic substrate.

19. The composition of claim 18, further comprising (i) a bile salt and (ii) a surfactant or lipid source.

20. The composition of claim 18, further comprising propylene glycol.
